(19) United States
(12) Patent Application Publication
(10) Pub. No.: US 2025/0261896 A1
(43) Pub. Date: Aug. 21, 2025

(54) SYSTEM AND METHOD OF CREATING CURATIVE GAMES

(71) Applicant: EMAZELABS LTD., Tzur Yitzhak (IL)

(72) Inventors: Omer HIRSH, Tzur Yitzhak (IL); Barak AVIV, Tzur Yitzhak (IL)

(21) Appl. No.: 19/056,923

(22) Filed: Feb. 19, 2025

Related U.S. Application Data
(60) Provisional application No. 63/555,114, filed on Feb. 19, 2024.

(51) Int. Cl. A61B 5/372; A61B 5/00; A61B 5/378; A61B 5/38; A61M 21/00; G16H 10/20; G16H 50/20

(52) U.S. Cl. CPC A61B 5/372; A61B 5/378; A61B 5/38; A61B 5/7267; A61M 21/00; G16H 10/20; G16H 50/20; A61M 2021/0027; A61M 2021/0044; A61M 2205/502; A61M 2230/10

(57) ABSTRACT

A system and a method of producing a curative game by at least one processor may include obtaining a brain map data structure, associating specific brain regions with respective, audiovisual stimuli; receiving an expert diagnosis of a neurological impairment of a subject; based on the expert diagnosis, prompting the subject to engage in at least one evaluation activity that corresponds to the neurological impairment, via a user interface (UI); assessing a functionality of one or more brain regions of the subject based on the evaluation activity; selecting one or more brain regions based on the assessed functionality; and creating at least one audiovisual, curative game designed to stimulate the selected brain region based on the brain map data structure.

### CROSS REFERENCE TO RELATED APPLICATIONS

This application claims the benefit of U.S. Patent Application No. 63/555,114, filed Feb. 19, 2024, and titled “SYSTEM AND METHOD OF CREATING CURATIVE GAMES”, which is hereby incorporated by reference in its entirety.

### FIELD OF THE INVENTION

The present invention relates generally to the technological field of assistive diagnostics treatment. More specifically, the present invention relates to systems and methods of creating curative games, for diagnosing and treating neurological impairments.

### BACKGROUND OF THE INVENTION

Neurological impairments, encompassing a spectrum of disorders affecting various regions of the brain, impose significant challenges on affected individuals and their families. With a growing prevalence globally, driven by various factors including lifestyle changes and environmental influences, these conditions lead to diminished quality of life, and socioeconomic burdens. Challenges in accurately diagnosing and effectively treating neurological impairments, especially in children, persist despite advances in medical science. Innovative approaches are needed to improve early detection, personalize treatment strategies, and enhance patient outcomes.

### SUMMARY OF THE INVENTION

As elaborated herein, embodiments of the invention may include a method and system for producing curatives game based on an iterative, rehabilitative neuroplastic process.

Embodiments of the invention may include a method of producing a curative game by at least one processor. According to some embodiments, the at least one processor may be configured to obtain a brain map data structure, associating specific brain regions with respective, audiovisual stimuli; receive an expert diagnosis of a neurological impairment of a subject; based on the expert diagnosis, prompt the subject to engage in at least one evaluation activity that corresponds to the neurological impairment, via a user interface (UI); assess, or calculate a functionality of one or more brain regions of the subject based on the evaluation activity; select one or more brain regions based on the assessed functionality; and create at least one audiovisual, curative game designed to stimulate the selected brain region based on the brain map data structure.

According to some embodiments, the at least one processor may prompt the subject to engage in an evaluation activity by presenting a questionnaire to the subject via the UI; obtaining the subject's response to the questionnaire via the UI; assessing the functionality of the one or more brain regions of the subject based on the subject's response; and personalizing the brain map to reflect the assessed functionality of one or more brain regions of the subject.

According to some embodiments, the at least one processor may assess the brain functionality of the one or more brain regions in an iterative process. Each iteration of this process may include prompting the subject to engage in an evaluation activity; assessing a functionality of one or more brain regions of the subject based on outcome of the evaluation activity; updating the brain map data structure, based on the assessed functionality, thereby producing a personalized brain map data structure; and employing a decision tree, to select an evaluation activity for a subsequent iteration, based on the personalized brain map data structure.

Additionally, or alternatively, the at least one processor may be configured to prompt the subject to engage in an evaluation activity by presenting evaluation game to the subject via the UI; monitoring the subject's playing, to extract a value of at least one evaluation game performance parameter; assessing the functionality of the one or more brain regions of the subject based on the at least one evaluation game performance parameter; and personalizing the brain map to reflect the assessed functionality of one or more brain regions of the subject.

According to some embodiments, the at least one processor may create a curative game by selecting, from a pool of game templates, a game template that corresponds to the neurological impairment; and adapting at least one game parameter of the selected game template, so as to stimulate the selected brain region, based on the brain map data structure.

According to some embodiments, the at least one processor may create the curative game by an iterative process. Each iteration of the iterative process may include prompting the subject to play (e.g., via the UI) one or more iterations of at least one created game; monitoring the subject's playing, to extract a value of at least one game performance parameter in the one or more iterations; analyzing the game performance parameter values, to identify suboptimal functionality of the one or more selected brain regions; and updating the brain map data structure to reflect the identified suboptimal functionality of the one or more selected brain regions.

According to some embodiments, the at least one processor may identify suboptimal functionality of one or more brain regions of the subject (e.g., human patient) by determining a rate of change, across the one or more iterations, of function of a specific region of the one or more selected brain regions; and analyzing, or comparing the determined rate of change in view of a predetermined baseline rate of expected change, to identify the suboptimal functionality.

Additionally, or alternatively, the at least one processor may train, or obtain a pretrained machine learning (ML) based model; and infer the ML based model on (i) the one or more game performance parameters, and (ii) the expert diagnosis, to predict a refined diagnosis of the neurological impairment of the subject.

According to some embodiments, the predicted refined diagnosis may include, for example an identification, or prediction of a suboptimal functionality of one or more brain regions of the subject; an assessment (e.g., a score), representing functionality of a specific, associated brain region; a subclass or an evaluation of a severity of the diagnosed neurological impairment; a refined version of a functional region map and/or a refined version of a region stimulus map of the subject; a prognosis, or an expected change in the neurological impairment condition of the subject; a recommended treatment, such as a game template to be used with the subject, and the like.

Additionally, or alternatively, the at least one processor may train, or obtain a pretrained ML based model; infer the ML based model on the one or more game performance parameters, to predict suboptimal functionality of at least one brain region of the subject; and update the brain map data structure to reflect the predicted suboptimal functionality of the at least one brain region.

Additionally, or alternatively, the at least one processor may adjust at least one game parameter of the created game based on the brain map data structure, so as to stimulate the selected brain region of the identified suboptimal functionality.

Additionally, or alternatively, the at least one processor may select, from a pool of game templates, a specific game template that corresponds to the neurological impairment; and adapt at least one game parameter of the selected game template based on the brain map data structure, so as to stimulate the selected brain region of the identified suboptimal functionality.

Additionally, or alternatively, the at least one processor may determine a rate of change across the one or more iterations, of functionality of a specific region of the one or more selected brain regions; and infer an ML-based model on (i) the determined rate of change, and (ii) the expert diagnosis, to predict a refined diagnosis of the neurological impairment of the subject.

Additionally, or alternatively, the at least one processor may create the curative game by obtaining, or calculating at least one profile data element, representing a profile of the subject; obtaining, or calculating at least one medical data element, representing a medical history of the subject; and adapting a game parameter of the selected game template based on the at least one profile data elements and the at least one medical data element, so as to stimulate the selected brain region, according to the brain map data structure.

Embodiments of the invention may include a system for treating a neurological impairment of a subject. Embodiments of the system may include a non-transitory memory device, wherein modules of instruction code are stored, and at least one processor associated with the memory device, and configured to execute the modules of instruction code.

Upon execution of the modules of instruction code, the at least one processor may be configured to: obtain a brain map data structure, associating specific brain regions with respective, audiovisual stimuli; receive an expert diagnosis of a neurological impairment of a subject; based on the expert diagnosis, prompt the subject to engage in at least one evaluation activity that corresponds to the neurological impairment, via a UI; based on the evaluation, produce an assessment of functionality of one or more brain regions of the subject; select one or more brain regions based on said assessment; create at least one audiovisual, curative game designed to stimulate the selected brain region based on the brain map data structure; and prompt the subject to play the created game via the UI.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a block diagram, depicting a computing device which may be included in a system for creating curative games for diagnosing and treating neurological impairments, according to some embodiments;

FIG. 2 is a block diagram, depicting a system for creating curative games according to some embodiments of the invention;

FIG. 3 is a block diagram, elaborating aspects of functionality of the system for creating curative games, according to some embodiments of the invention;

FIG. 4A and 4B are tables elaborating aspects of a brain map data structure, according to some embodiments of the invention;

FIG. 5 is a table elaborating examples of brain region-specific classifiers which may be included in some embodiments of the invention;

FIGS. 6A and 6B present data that may be accumulated from a subject, representing one use case of an embodiment of the invention; and

FIG. 7 is a flow diagram, depicting a method of creating curative games according to some embodiments of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

One skilled in the art will realize the invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention. Some features or elements described with respect to one embodiment may be combined with features or elements described with respect to other embodiments. For the sake of clarity, discussion of same or similar features or elements may not be repeated.

Although embodiments of the invention are not limited in this regard, discussions utilizing terms such as, for example, “processing,” “computing,” “calculating,” “determining,” “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium that may store instructions to perform operations and/or processes.

Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term “set” when used herein may include one or more items.

Unless explicitly stated, the method embodiments described herein are not constrained to a particular order or sequence. Additionally, some of the described method embodiments or elements thereof can occur or be performed simultaneously, at the same point in time, or concurrently.

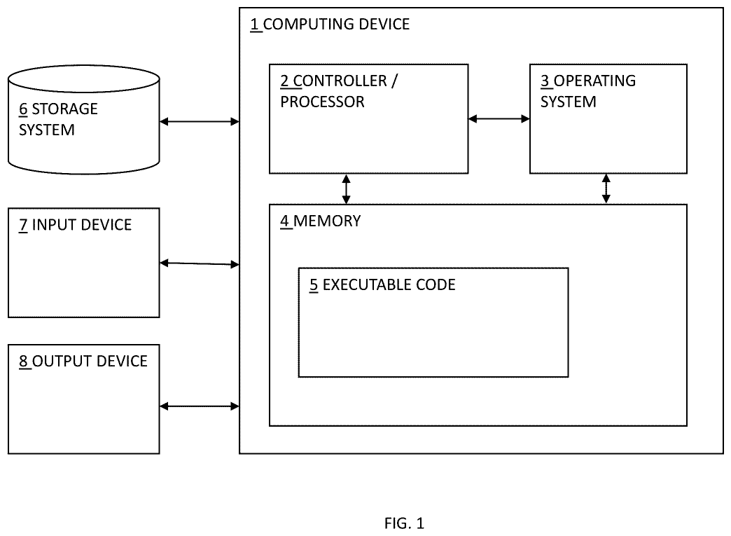

Reference is now made to FIG. 1, which is a block diagram depicting a computing device, which may be included within an embodiment of a system for creating curative games for diagnosing and treating neurological impairments, according to some embodiments.

Computing device 1 may include a processor or controller 2 that may be, for example, a central processing unit (CPU) processor, a chip or any suitable computing or computational device, an operating system 3, a memory 4, executable code 5, a storage system 6, input devices 7 and output devices 8. Processor 2 (or one or more controllers or processors, possibly across multiple units or devices) may be configured to carry out methods described herein, and/or to execute or act as the various modules, units, etc. More than one computing device 1 may be included in, and one or more computing devices 1 may act as the components of, a system according to embodiments of the invention.

Operating system 3 may be or may include any code segment (e.g., one similar to executable code 5 described herein) designed and/or configured to perform tasks involving coordination, scheduling, arbitration, supervising, controlling or otherwise managing operation of computing device 1, for example, scheduling execution of software programs or tasks or enabling software programs or other modules or units to communicate. Operating system 3 may be a commercial operating system. It will be noted that an operating system 3 may be an optional component, e.g., in some embodiments, a system may include a computing device that does not require or include an operating system 3.

Memory 4 may be or may include, for example, a Random-Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short term memory unit, a long term memory unit, or other suitable memory units or storage units. Memory 4 may be or may include a plurality of possibly different memory units. Memory 4 may be a computer or processor non-transitory readable medium, or a computer non-transitory storage medium, e.g., a RAM. In one embodiment, a non-transitory storage medium such as memory 4, a hard disk drive, another storage device, etc. may store instructions or code which when executed by a processor may cause the processor to carry out methods as described herein.

Executable code 5 may be any executable code, e.g., an application, a program, a process, task, or script. Executable code 5 may be executed by processor or controller 2 possibly under control of operating system 3. For example, executable code 5 may be an application that may create curative games as further described herein. Although, for the sake of clarity, a single item of executable code 5 is shown in FIG. 1, a system according to some embodiments of the invention may include a plurality of executable code segments similar to executable code 5 that may be loaded into memory 4 and cause processor 2 to carry out methods described herein.

Storage system 6 may be or may include, for example, a flash memory as known in the art, a memory that is internal to, or embedded in, a micro controller or chip as known in the art, a hard disk drive, a CD-Recordable (CD-R) drive, a Blu-ray disk (BD), a universal serial bus (USB) device or other suitable removable and/or fixed storage unit. Data pertaining to a human subject or patient may be stored in storage system 6 and may be loaded from storage system 6 into memory 4 where it may be processed by processor or controller 2. In some embodiments, some of the components shown in FIG. 1 may be omitted. For example, memory 4 may be a non-volatile memory having the storage capacity of storage system 6. Accordingly, although shown as a separate component, storage system 6 may be embedded or included in memory 4.

Input devices 7 may be or may include any suitable input devices, components, or systems, e.g., a detachable keyboard or keypad, a mouse and the like. Output devices 8 may include one or more (possibly detachable) displays or monitors, speakers and/or any other suitable output devices. Any applicable input/output (I/O) devices may be connected to Computing device 1 as shown by blocks 7 and 8. For example, a wired or wireless network interface card (NIC), a universal serial bus (USB) device or external hard drive may be included in input devices 7 and/or output devices 8. It will be recognized that any suitable number of input devices 7 and output device 8 may be operatively connected to Computing device 1 as shown by blocks 7 and 8.

A system according to some embodiments of the invention may include components such as, but not limited to, a plurality of central processing units (CPU) or any other suitable multi-purpose or specific processors or controllers (e.g., similar to element 2), a plurality of input units, a plurality of output units, a plurality of memory units, and a plurality of storage units.

The term neural network (NN) or artificial neural network (ANN), e.g., a neural network implementing a machine learning (ML) or artificial intelligence (AI) function, may be used herein to refer to an information processing paradigm that may include nodes, referred to as neurons, organized into layers, with links between the neurons. The links may transfer signals between neurons and may be associated with weights. A NN may be configured or trained for a specific task, e.g., pattern recognition or classification. Training a NN for the specific task may involve adjusting these weights based on examples. Each neuron of an intermediate or last layer may receive an input signal, e.g., a weighted sum of output signals from other neurons, and may process the input signal using a linear or nonlinear function (e.g., an activation function). The results of the input and intermediate layers may be transferred to other neurons and the results of the output layer may be provided as the output of the NN. Typically, the neurons and links within a NN are represented by mathematical constructs, such as activation functions and matrices of data elements and weights. At least one processor (e.g., processor 2 of FIG. 1) such as one or more CPUs or graphics processing units (GPUs), or a dedicated hardware device may perform the relevant calculations.

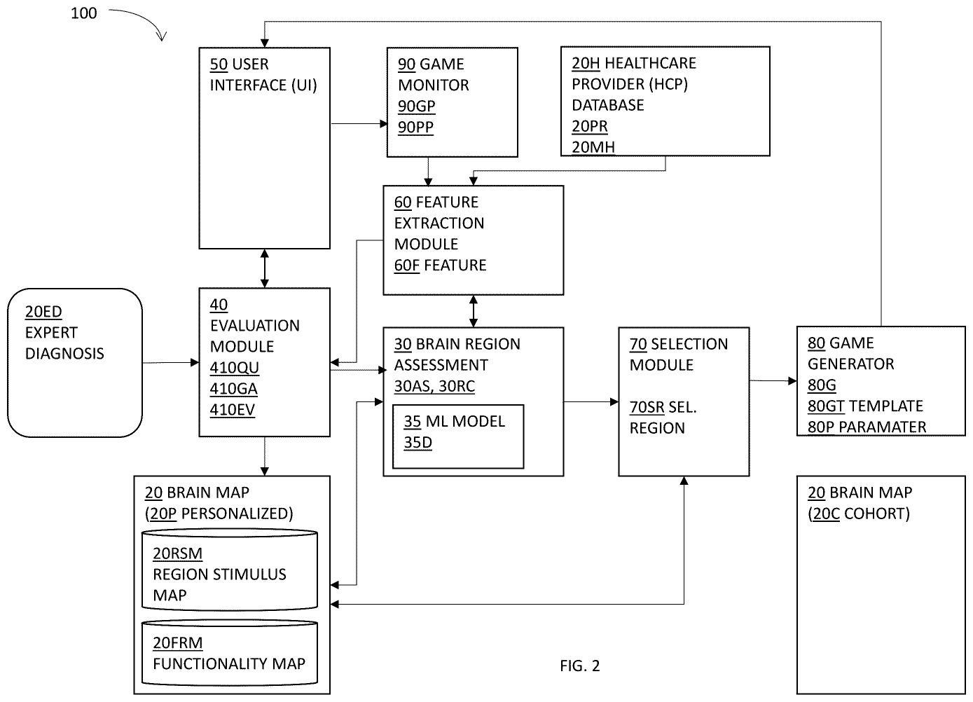

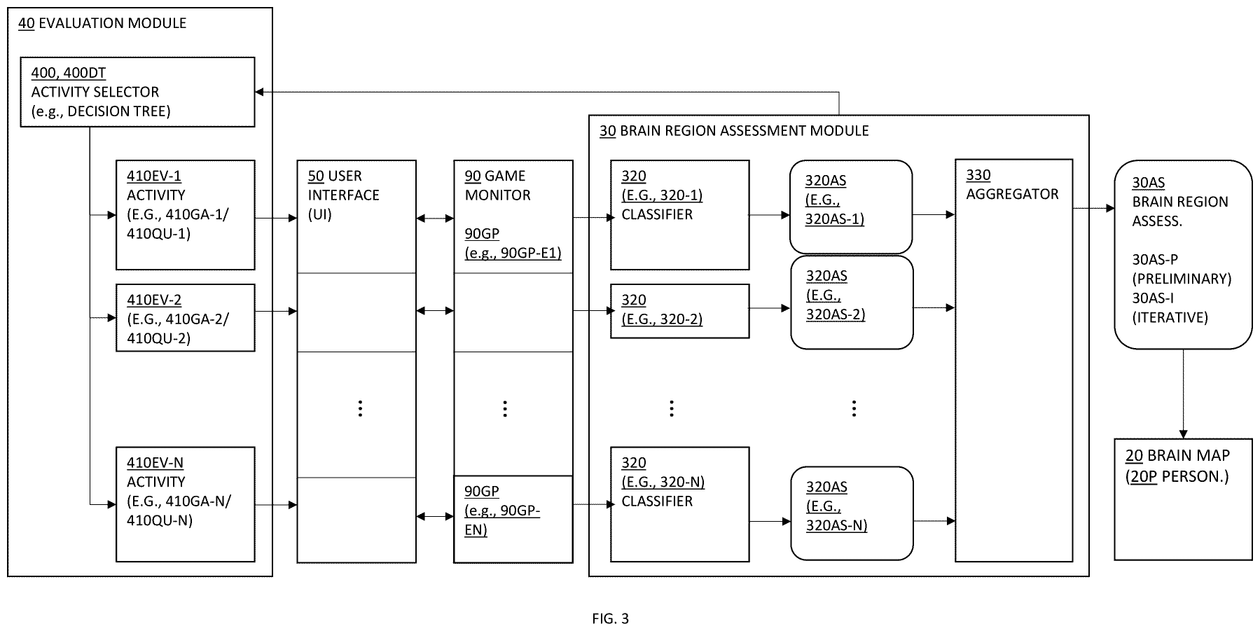

Reference is now made to FIG. 2, which depicts a system 100 for creating curative games, for diagnosing and treating neurological impairments, according to some embodiments. Reference is also made to FIG. 3, which shows aspects of functionality of system 100, according to some embodiments of the invention.

According to some embodiments of the invention, system 100 may be implemented as a software module, a hardware module, or any combination thereof. For example, system 100 may be or may include a computing device such as element 1 of FIG. 1, and may be adapted to execute one or more modules of executable code (e.g., element 5 of FIG. 1) to create curative games as further described herein.

As shown in FIG. 2, arrows may represent flow of one or more data elements to and from system 100 and/or among modules or elements of system 100. Some arrows have been omitted in FIG. 2 for the purpose of clarity.

As shown in FIG. 2, system 100 may receive, e.g., from at least one computing device or database, a brain map data structure 20. According to some embodiments, brain map data structure 20 (or “brain map 20”, for short) may include one or more data structures such as tables or maps, that may associate specific brain regions with respective, audiovisual stimuli and/or high-level functionality.

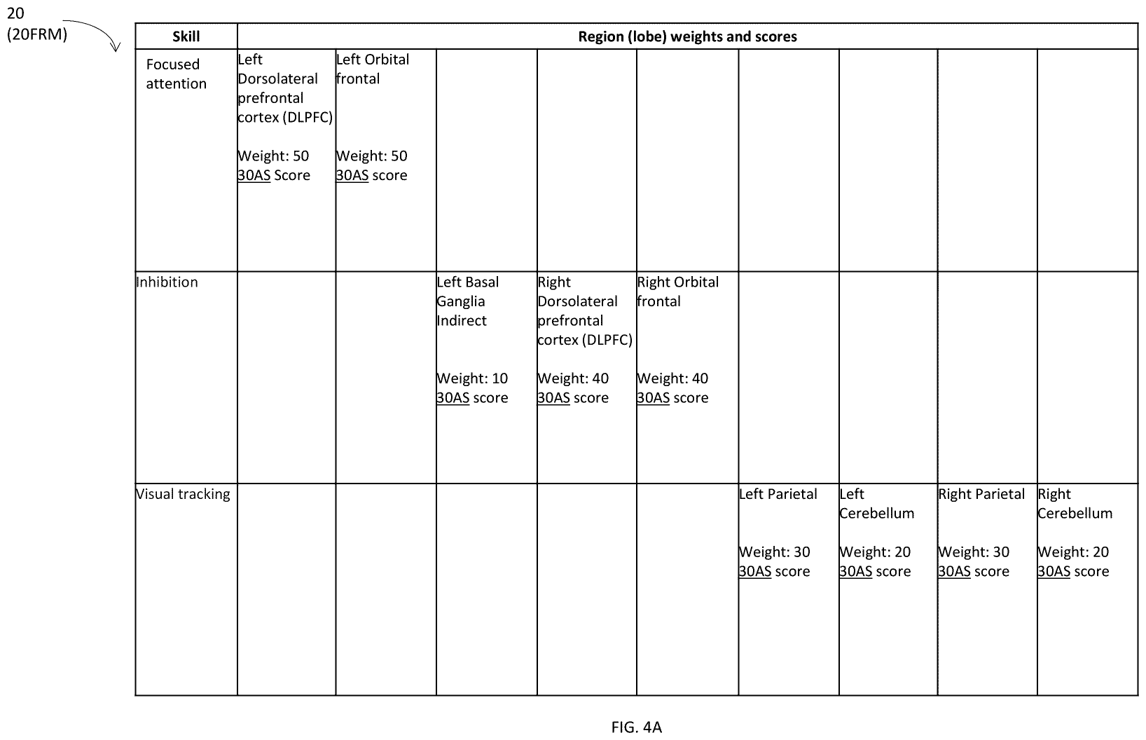

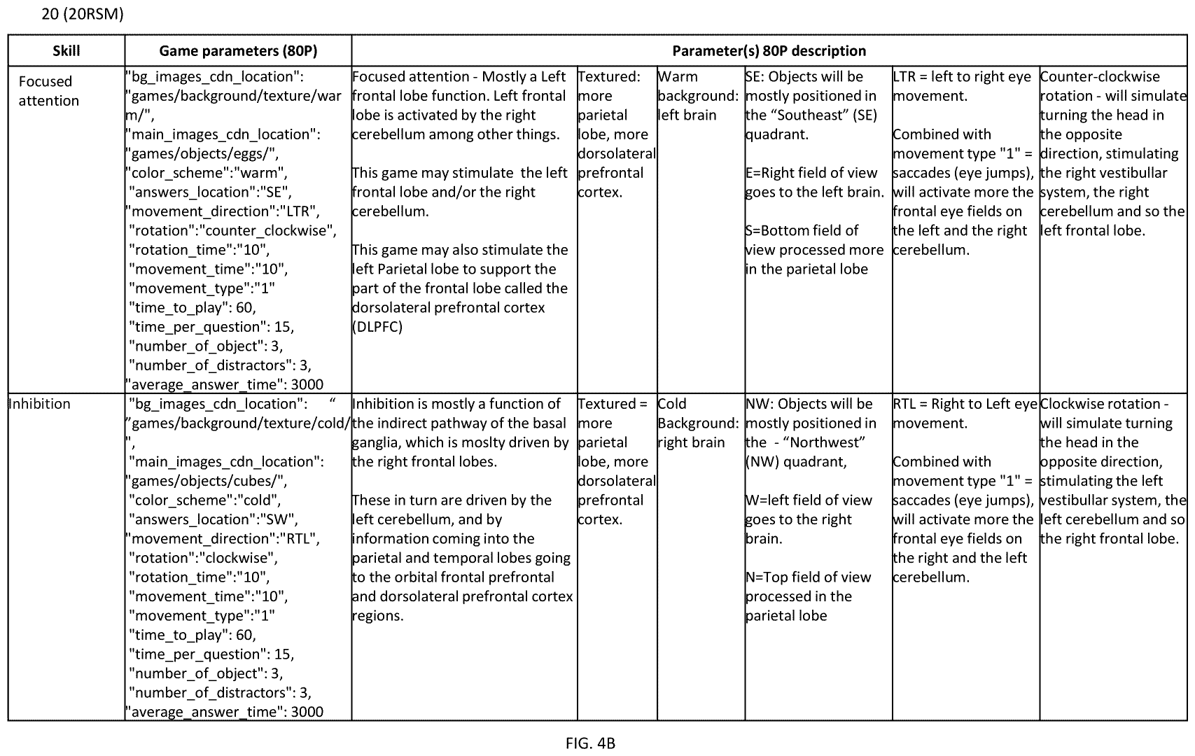

Reference also made to FIGS. 4A and 4B which are tables elaborating aspects of a brain map data structure 20, according to some embodiments of the invention.

According to some embodiments, brain map 20 may include, or be arranged as a functional region map data structure 20FRM that may associate between specific high-level functions (e.g., reading) and specific, respective regions in the brain that are incorporated in that functionality (in this example, the left hemisphere frontal, temporal and parietal regions).

Additionally, or alternatively, functional region map data structure 20FRM may indicate an extent, or weight (e.g., in time and space) of participation of specific brain regions in a high-level function or task.

Pertaining to the example of FIG. 4A, the task of focused attention may be weighted, or divided in a ratio of [50:50] between the Left Dorsolateral prefrontal cortex (DLPFC) and Left Orbital Frontal (LOF) lobe.

In another example, the task of inhibition may be weighted, or divided in a ratio of [10:40:40:10] between the Left Basal Ganglia Indirect, the Right DLPFC, the Right Orbital frontal lobe, and the Right Basal Ganglia Indirect.

In yet another example, the task of visual tracking may be weighed, or divided at a ratio of [30:20:30:20] between the Left Parietal lobe, Left Cerebellum Right Parietal lobe and Right Cerebellum lobes.

Additionally, or alternatively, brain map 20 may include a Region Stimulus Map (RSM) 20RSM, that may associate specific brain regions with respective, audiovisual stimuli. In other words, region stimulus map 20RSM may indicate amplitude and/or extent (e.g., in time and space) of an effect of a specific stimulus (e.g., image and/or audio) that may be presented to a subject via a User Interface (UI) 50 (such as devices 7, 8 of FIG. 1), on specific brain regions of the subject.

Pertaining to the example of FIG. 4B, in order to evaluate a specific skill or task, region stimulus map 20RSM of brain map 20 may associate this skill with a list of predetermined game parameter 80P values. The list of game parameter values 80P may be designed to stimulate the brain regions associated with the specific task or activity.

For example, to evaluate a skill of focused attention, brain map 20 (20RSM) may specify values of game parameters 80P such as a texture (adapted to stimulate the parietal lobe and dorsolateral prefrontal cortex), a background colour (warm background stimulating the left hemisphere), a type of movement of presented objects (left to right saccadic movement to stimulate the left and right Cerebellum), position of presented objects (“Southeast” (SE) quadrant, to stimulate the left Parietal lobe), and the like.

It may be appreciated that the examples provided in FIGS. 4A and 4B are brought for clarification, and are neither limiting, nor exhaustive. Both parts of brain map 20, e.g., functional region map data structure 20FRM and region stimulus map 20RSM, may include additional entries, pertaining to additional cognitive functionalities and additional brain regions.

As shown in FIG. 2, system 100 may receive, e.g., via input device 7 of FIG. 1 an expert diagnosis 20ED of a neurological impairment of a subject. For example, expert diagnosis 20ED may include indication of a cognitive or behavioural neurological impairment, such as Attention-Deficit/Hyperactivity Disorder (ADHD), Autistic Spectrum Disorder (ASD), Dyslexia, Sensory Processing Disorder (SPD), and the like.

In another example, expert diagnosis 20ED may include indication of a motoric neurological impairment, such as Dysgraphia, Developmental Coordination Disorder (DCD), stuttering, and the like.

As shown in FIG. 2, system 100 may include an initial evaluation module 40 (or “evaluation module 40”, for short). According to some embodiments, initial evaluation module 40 may be configured to prompt the subject to engage, e.g., via UI 50 in at least one evaluation activity 410EV. Evaluation activity 410EV may correspond to one or more specific neurological impairments based on, or expressed by the initial expert diagnosis 20ED.

Initial expert diagnosis 20ED may include an indication of neurological impairment such as a reading disability (e.g., developmental dyslexia, acquired dyslexia, hyperlexia, etc.) of a specific subject. Initial evaluation module 40 may adapt, or select evaluation activity 410EV to evaluate specific, personal characteristics of that subject according to initial expert diagnosis 20ED.

As elaborated herein, evaluation module 40 may produce an evaluation activity 410EV that includes one or more questionnaires 410QU (denoted 410QU-1, . . . , 410QU-N) or forms. Questionnaires 410QU may be adapted to obtain parameters or characteristics describing the subject's neurological impairment. Evaluation module 40 may present evaluation activity 410EV (e.g., questionnaire 410QU) via UI 50, to obtain the subject's response to the questionnaire via UI 50. The subject's response may include, for example structured, or semi-structured data representing the subject's personal characteristics of the neurological impairment included in the initial expert diagnosis 20ED.

In another example, evaluation module 40 may produce an evaluation activity 410EV that includes one or more evaluation games 410GA (denoted 410GA-1, . . . , 410GA-N), adapted to evaluate the subject's characteristics of the specific neurological impairment.

Evaluation module 40 may present evaluation activity 410EV (e.g., evaluation game(s) 410GA) via UI 50 to the subject. Evaluation module 40 may collaborate with a monitoring module 90, configured to monitor the subject's activity (e.g., playing), to extract a value of at least one respective evaluation game performance parameter 90GP (denoted 90GP-E1, . . . , 90GP-EN), representing for example, the subject's response time, success rate, overall success or failure, and the like, as elaborated herein.

Evaluation module 40 may collaborate with a brain assessment module 30, adapted to assess the functionality of the one or more brain regions of the subject, based on outcome of the one or more evaluation activities 410EV (e.g., the subject's response to questionnaire(s) 410QU-1, . . . , 410QU-N) and/or evaluation game performance parameter 90GP (denoted 90GP-E1, . . . , 90GP-EN). As elaborated herein, Brain assessment module 30 may assess the functionality of the one or more brain regions based on evaluation activity.

According to some embodiments, brain assessment module 30 may calculate an assessment 30AS representing overall severity of the subject's underlying neurological impairment, based on brain map data structure 20. This assessment of brain region functionality may be performed iteratively, e.g., by a cascade of classifier modules 320 (e.g., 320-1, . . . , 320-N). Each iteration may include (a) selection of an evaluation activity 410EV by an activity selection module 400, (b) assessing (e.g., 320AS-1, 320AS-2, etc.) functionality of an interim subset of brain regions by a respective classifier 320, and (c) prompting the subject to engage in a subsequent evaluation activity 410EV, based on the results of the current iteration.

Additionally, or alternatively, assessment module 30 may update the brain map data structure 20 (e.g., 20FRM), based on (e.g., to include) the assessed functionality scores 320AS, thereby personalizing an interim version of brain map data structure 20, according to the underlying diagnosed impairment 20ED. In a subsequent iteration, system 100 may prompt the subject to engage in a subsequent evaluation activity 410EV, based on the personalized brain map data structure.

The iterative assessment process may end by aggregating assessment results or scores 320AS (e.g., 320AS-1, . . . , 320AS-N), thereby providing an overall assessment 30AS of brain regions.

Additionally, or alternatively, the iterative assessment process may end with updating brain map 20 (20FRM) to reflect, or include assessment scores 30AS, as shown in FIG. 4A, thereby producing an initial baseline (e.g., prior to treatment), personalized, brain map 20, representing functionality of brain regions of the specific subject.

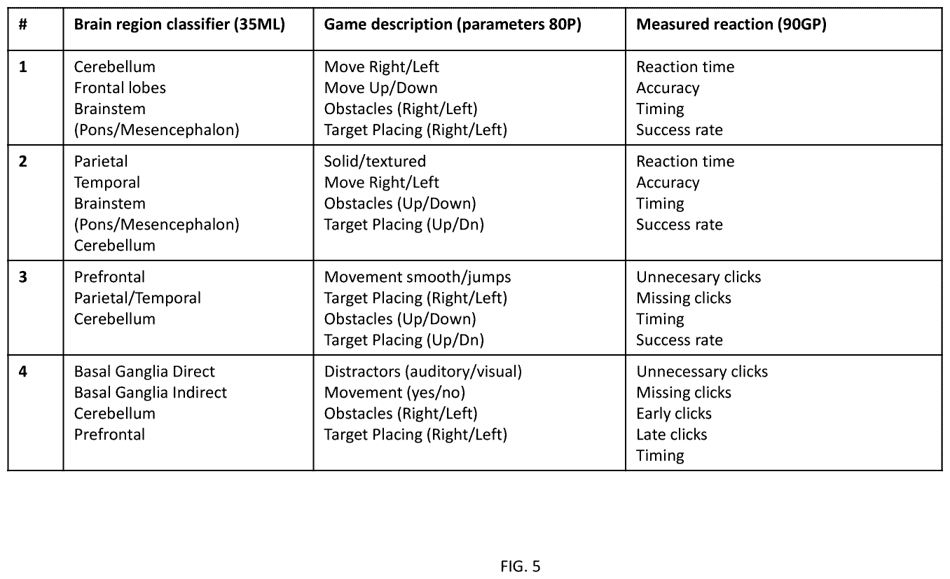

Reference is now made to FIG. 5, which is a table elaborating examples of brain region-specific classifiers 320, which may be included in some embodiments of the invention. As shown in this example, each brain region-specific classifier 320 may be associated with respective game parameters 80P, specifically selected to stimulate the respective brain region, as elaborated herein. Additionally, each brain region-specific classifier 320 may be associated with respective reaction measurements 90GP, that correspond to activities 410EV. Reaction measurements 90GP have been experimentally found to be prominent in the assessment or score 30AS of the respective brain region functionality.

For example, a subject's neurological impairment may be a reading impairment, such as dyslexia. As known in the art, a skill that is required for reading includes the ability to perform visual tracking. As shown in FIG. 4A, and based on brain map data structure 20, this ability is governed by the left and right Parietal lobes, as well as the left and right Cerebellum.

As shown in FIG. 5, activity selection module 400 may therefore select a first activity 410EV (e.g., evaluation game 410GA-1) such as game #4, that is adapted to stimulate, and assess a first subset of brain regions (e.g., the Cerebellum, but not the Parietal lobes), and then proceed to a second activity 410EV (e.g., evaluation game 410GA-2) such as game #1, that is adapted to stimulate, and assess a second subset of brain regions (e.g., the Cerebellum, and the Parietal lobes).

During the first game 410GA-1 (#4), a first set of game parameters 80P may be applied (e.g., using auditory and visual distractors), and a first set of user reactions may be measured or counted (e.g., counting unnecessary mouse-clicks missing mouse-clicks and early (premature) mouse-clicks), to calculate score 320AS (e.g., higher score 320AS when there are less unnecessary or premature mouse clicks).

During the second game 410GA-2 (#1), a second set of game parameters 80P (e.g., moving a target up and down) may be applied, and a second set of user reactions may be measured (e.g., reaction time, hit-accuracy and overall target-hit success rate), to calculate score 320AS (e.g., higher score for 320AS) for lower reaction time.

As explained herein, each activity 410EV may stimulate multiple brain regions. Activity selection module 400 may therefore employ at least one rule-based decision tree 400DT, that may allow separation of functionality assessment to individual brain regions.

In other words, decision tree 400DT may pertain to a specific, underlying, expert-diagnosed impairment 20ED. Decision tree 400DT may be specifically configured to select evaluation activities (i) hierarchically, according to brain region significance, to allow efficient analysis using a minimal number of games or sessions, and (ii) have low correlation between their respectively stimulated brain regions, to allow the required separation of region-specific assessment scores 320AS.

As explained herein, brain assessment module 30 may calculate an assessment or score 30AS for functionality of one or more (e.g., each) brain region that is associated with the underlying neurological impairment, in accordance with brain map data structure 20. Brain assessment module 30 may subsequently personalize the brain map data structure 20 of a specific subject to reflect the assessed functionality of one or more brain regions of that subject, as shown in FIG. 4A.

Pertaining to the example of FIG. 4A, initial expert diagnosis 20ED may include indication of a focused attention impediment in a subject. Brain assessment module 30 may analyze performance 90GP of the subject in playing a game 410GA that is specifically designed to evaluate the subject's focused attention. Brain assessment module 30 may thereby assess scores 30AS of specific regions in the subject's brain (Left DLPFC and/or Left Orbital Frontal Lobe), and personalize brain map 20 to reflect these scores.

Additionally, or alternatively, neurological impairment may include a high-level neurological impairment, such as a reading disability. Evaluation activity 410EV may produce an evaluation activity 410EV that is adapted to evaluate functionality of reading-related regions in the brain. Following performance of the evaluation activity 410EV on UI 50, evaluation activity 410EV may collaborate with assessment module 30 to produce an assessment 30AS or a score of functionalities of one or more brain regions of the subject, based on evaluation activity 410EV.

According to some embodiments, evaluation module 40 may collaborate with assessment module 30 to produce assessment 30AS as a personalized, baseline version of brain map 20.

For example, as shown in FIG. 4B, region stimulus map 20RSM may associate specific brain functions and/or brain regions of a subject with respective, audiovisual stimuli. Evaluation module 40 may produce assessment 30AS as a personalized (i.e., pertaining to the specific subject), baseline version of region stimulus map 20RSM, e.g., representing an initial setting of one or more game parameters (e.g., initial appearance, location, or speed of objects in the game) that may evolve over time (e.g., as part of a treatment trajectory).

In another example, as shown in FIG. 4A, evaluation module 40 may produce assessment 30AS as an initial, personalized, baseline version of a functional region map data structure 20FRM, that may evolve over time.

The term “initial” may be used in this context to indicate that assessment module 30 may repeat the assessment process in a plurality of iterations, to produce further assessments 30AS e.g., in response to further activity of the subject (e.g., games 410GA/80G played by the subject), as further elaborated herein. The term “personal” may be used in this context to indicate that assessment 30AS may pertain to a specific, relevant subject or patient of diagnosis 20ED, and may be compared to a “normal” or “cohort” assessments 30AS that may represent a population of subjects. The term “baseline” may be used in this context to indicate that assessments 30AS may present a starting point to follow evolution (e.g., improvement or degradation) of the subject's condition, following treatment.

As shown in FIG. 2, system 100 may include a selection module 70, adapted to select one or more brain regions 70SR based on assessment 30AS.

For example, selection module 70 may receive a cohort functional region map 20FRM, representing mean values of a population of gender and age-similar subjects. Selection module 70 may compare a functional region map 20FRM of a personalized brain map 20 (denoted 20P, pertaining to a specific subject) with a functional region map 20FRM of a cohort brain map 20 (denoted 20C, and pertaining to a cohort of subjects). Selection module 70 may select 70SR at least one brain region of the subject that may be characterized by an insufficient score 30AS, among brain regions participating in a predetermined function of interest (e.g., reading).

Additionally, or alternatively, selection module 70 may receive a region stimulus map 20RSM of a cohort brain map 20 (denoted 20C, representing mean values of a population of gender and age-similar subjects). Selection module 70 may compare a region stimulus map 20RSM of a personalized brain map 20 (pertaining to a specific subject) with the cohort region stimulus map 20RSM, to identify and select at least one brain region 70SR of the subject that may be characterized by insufficient amplitude and/or extent (e.g., in time and space) for stimulating the low-scoring 30AS brain region of that subject.

As shown in FIG. 2, system 100 may include a game generation module 80, adapted to create at least one audiovisual (e.g., containing audio and/or visual content) curative game 80G. Curative game 80G may be designed to stimulate the selected brain region 70SR based on the subject's brain map data structure 20, e.g., based on scores 30AS in functional map 20FRM and/or based on the association of brain regions to audiovisual stimuli in region stimulus map 20RSM.

Additionally, or alternatively, game generation module 80 may be configured to produce a pool of curative games 80G. Games 80G of the pool may correspond to the neurological impairment of expert diagnosis 20ED (e.g., to treat a reading impairment).

The pool of curative games 80G may differ by at least one audiovisual game parameter 80P (e.g., having different colors, sounds, speed, etc.). Game generation module 80 may subsequently collaborate with selection module 70 to select a specific curative game 80G, designed to stimulate the selected brain region 70SR based on the subject's brain map 20.

Additionally, or alternatively, game generation module 80 may select from a pool of game templates 80GT, a game template that corresponds to the neurological impairment of expert diagnosis 20ED (e.g., directed to treating a reading impairment). Game generation module 80 may subsequently adapt at least one game parameter 80P of the selected game template 80GT, so as to stimulate the selected brain region 70SR, according to the subject's brain map 20.

For example, game parameter 80P may include characteristics of movement of one or more objects in curative game 80G, e.g., saccadic movement, linear movement, rotational movement, etc. Game generation module 80 may adapt game parameter 80P, to change (e.g., change a movement direction and type), to activate or stimulate a relevant brain region, according to brain map 20 (20RSM). For example, a game 80G that includes game parameters 80P such as smooth pursuit from left to right may be associated with parietal and temporal lobes on the right hemisphere, while a game 410GA/80G that includes game parameters 80P such as quick jumps from left to right may be associated with stimulation of the frontal lobes on the left hemisphere.

According to some embodiments, system 100 may use UI 50 to prompt the subject to play one or more iterations of at least one created game 80G, and monitor the playing by at least one game monitoring module 90. Monitoring module 90 may thereby extract a value of at least one game performance parameter 90GP in the one or more game iterations.

Game performance parameters 90GP may include, for example, a measurement of reaction time of the subject, a number of actions (e.g., mouse clicks) the subject has performed (e.g., in comparison to a number of required actions), a total number of completed games (e.g., as portion of a number of games defined by a provider), a number of daily sessions compared to a predefined number, and the like.

Additionally, or alternatively, game 410GA/80G may be designed to determine a user's ability to follow a moving target, e.g., using a touch screen or mouse curser. Game performance parameters 90GP may include a measurement of accuracy (e.g., how close to the target did the user click, or a difference between a position of a presented object and a point of a subject's contact), or the subject's capability to visually follow the target (e.g., using an eye movement tracking device). Additionally, or alternatively, game 410GA/80G may include distracting objects, and performance parameters 90GP may include comparison of success and/or accuracy in relation to the level of distraction (e.g., number and/or movement of distracting objects).

Additionally, or alternatively, game 410GA/80G may prompt the subject to respond to a task or question. Game performance parameters 90GP may include a success rate (e.g., correct vs incorrect responses, number of correct as percentage of a total number of responses, etc.), a time required by the subject to answer, a number of consecutive correct answers, and the like.

Assessment module 30 may subsequently analyze game performance parameter values 90GP to recalculate assessment 30AS, and/or update brain map 20 (e.g., 20RSM and/or map 20FRM). In other words, assessment module 30 may analyze game performance parameter values 90GP to determine or identify suboptimal functionality of the one or more selected brain regions 70SR.

For example, game monitoring module may produce a standardized performance profile 90PP, representing a metric (e.g., a mean) of performance parameters 90GP pertaining to a cohort of gender and age-similar subjects. Assessment module 30 may then analyze performance parameters 90GP of a specific subject in playing curative game 80G, in view of standardized performance profile 90PP, to identify suboptimal functionality of the one or more selected brain regions 70SR.

In another example, assessment module 30 may calculate a rate of change 30RC, across the one or more game 80G iterations, of a function of a specific selected brain region(s) 70SR, of a specific subject, as represented by one or more consecutively generated functional region maps 20FRM. Assessment module 30 may analyze the determined rate of change 30RC in view of a predetermined baseline rate of change 30RC (e.g., an expected rate of change 30RC).

Assessment module 30 may thereby determine suboptimal functionality of a specific brain region 70SR according to this analysis. For example, when a rate of change 30RC in functionality of a brain region of the subject (as reflected by functional region maps 20FRM) when playing curative game 80G falls short of an expected, baseline rate of change 30RC, then assessment module 30 may determine that the relevant brain region is characterized by suboptimal functionality.

Assessment module 30 may then collaborate with game generation module 80 to adjust at least one game parameter 80P of the curative game 80G, so as to stimulate the selected brain region 70SR, according to the brain map 20. System 100 may thereby dynamically, and interactively improve the functionality of the relevant brain region 70SR.

Additionally, or alternatively, game generation module 80 may collaborate with selection module 70, to select from a pool of game templates 80GT, a specific game template 80GT that corresponds to the neurological impairment of diagnosis 20ED based on the analysis of the brain functionality rate of change 30RC. Game generation module 80 may then adapt at least one game parameter 80P of the selected game template 80GT, so as to stimulate the selected brain region 70SR, in accordance with the functional region maps 20FRM of brain map 20.

According to some embodiments, the generation of curative games and analysis of the subject's performance may be an iterative, or repetitive process over time. Each iteration of the iterative process may include the steps of: (a) prompting the subject to play one or more iterations of at least one curative game 80G on UI 50; (b) monitoring the subject's playing, to extract a value of at least one game performance parameter 90GP in the one or more iterations; (c) analyzing the game performance parameter values, to identify suboptimal functionality of one or more selected brain regions 70SR; and (d) optionally updating brain map data structure 20, to reflect (or include) a current value, or assessment 30AS of the identified suboptimal functionality of the one or more selected brain regions 70SR.

According to some embodiments, at least one classification module 320 may be, or may include a machine-learning (ML) based model 35. In such embodiments, ML based model 35 may be dedicated to, or associated with one or more specific brain regions, and may be trained to predict, or assess functionality score 30AS of the associated brain region(s), based at least in part on game performance parameters 90GP, as elaborated herein.

Additionally, or alternatively, brain region assessment module 30 may include one or more machine-learning (ML)-based models 35 that may be trained to calculate, or predict a diagnosis 35D of one or more brain region(s). Diagnosis 35D may include, for example functionality score 30AS, representing functionality of the one or more brain regions.

Additionally, or alternatively, ML based model(s) 35 may be trained to predict a refined diagnosis 35D of the neurological impairment of the subject. For example, refined diagnosis 35D may include a subclass or an evaluation of a severity of the neurological impairment, defined by expert diagnosis 20ED.

Additionally, or alternatively, refined diagnosis 35D may include a refined version of a functional region map 20FRM and/or a refined version of a region stimulus map 20RSM of the subject.

Additionally, or alternatively, refined diagnosis 35D may include a prognosis of a neurological impairment of diagnosis 20ED, and/or an expected change in the neurological impairment condition of the subject.

Additionally, or alternatively, refined diagnosis 35D may include a recommended treatment, such as a game template 80GT to be used for creating new curative game 80G, that may be subsequently prompted to the subject.

As shown in FIG. 2, system 100 may obtain, e.g., from at least one Health Care Provider (HCP) computing device 20H, at least one profile data element 20PR. Profile data element 20PR may include, or represent profile information of a subject, including for example the subject's age, gender, ethnicity, and the like.

Additionally, or alternatively, system 100 may obtain (e.g., from computing device 20H) at least one medical data element 20MH, representing a medical history of the subject, such as previous diagnoses of the subject, hospitalizations events, and medication prescribed to the subject.

According to some embodiments, system 100 may include a feature extraction module 60, configured to receive an initial set of features, that may include, for example game performance parameters 90GP (from game monitoring module 90), medical data elements 20MH, profile data elements 20PR, and brain functionality rate of change 30RC.

System 100 may (e.g., during a training stage) provide ML model 35 with a plurality of feature vectors 60F, pertaining to a plurality of use-cases or subjects as input. System 100 may train ML model 35 by an appropriate training scheme (e.g., by a backward-propagation process), to predict refined diagnosis 35D as a function of the feature vectors 60F.

ML model 35 may subsequently (e.g., during an inference stage), receive a target feature vector 60F, pertaining to a specific use-case or subject of interest. ML model 35 may then predict refined diagnosis 35D as a function of target feature vector 60F, in accordance with its training.

Additionally, or alternatively, system 100 may obtain a trained ML based model 35, and infer ML model on (i) the one or more game performance parameters, and (ii) the expert diagnosis, to predict a refined diagnosis 35D of the neurological impairment of the subject.

Additionally, or alternatively, system 100 may infer ML based model 35 on the one or more game performance parameters 90GP, to produce a diagnosis 35D that may include prediction of suboptimal functionality of at least one brain region of the subject. System 100 may subsequently update the brain map data structure 20 of that subject to reflect the predicted suboptimal functionality of the at least one brain region.

Additionally, or alternatively, feature extraction module 60 may collaborate with ML model 35 to extract, from an initial set of features, a reduced set of features 60F that are most prominent for predicting refined diagnosis 35D. In a subsequent inference stage, system 100 may infer ML model 35 on the reduced set of features 60F, to predict refined diagnosis 35D.

For example, feature extraction module 60 may determine that a rate of change 30RC of function of a specific region of the one or more selected brain regions 70SR, across gaming iterations is a prominent feature for predicting refined diagnosis 35D. System 100 may subsequently infer ML based model 35 on (i) the determined rate of change 30RC, and (ii) the expert diagnosis 20ED, to predict a refined diagnosis 35D of the neurological impairment of the subject.

In another example, feature extraction module 60 may determine that at least one profile data element 20PR and/or at least one medical data element 20MH may be prominent for predicting refined diagnosis 35D. System 100 may subsequently infer ML based model 35 further on profile data element 20PR and/or medical data element 20MH to produce refined diagnosis 35D.

In yet another example, refined diagnosis 35D may include a recommendation for a specific game 80G and/or game template 80GT, to be used for treating the relevant neural impairment. In such embodiments, game generation module 80 may adapt at least one game parameter 80P of a selected game template 80GT based on at least one profile data element 20PR and/or medical data element 20MH, to stimulate the selected brain region 70SR, according to the brain map 20.

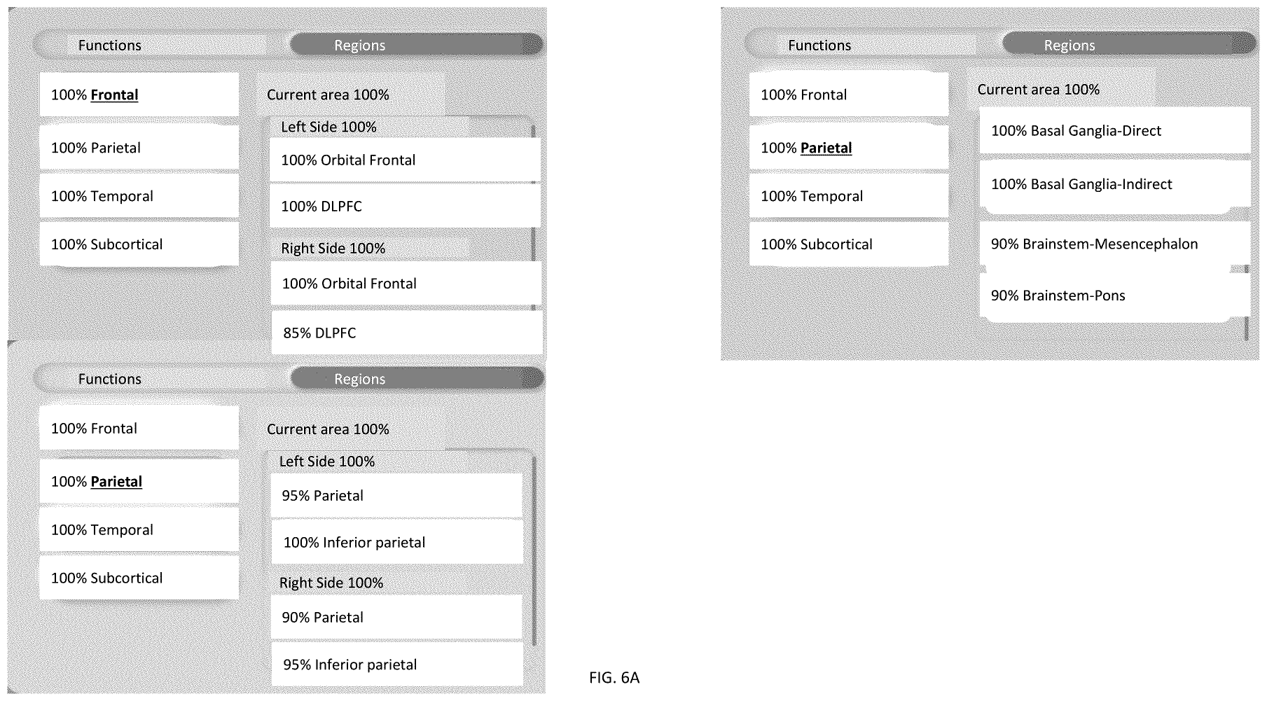

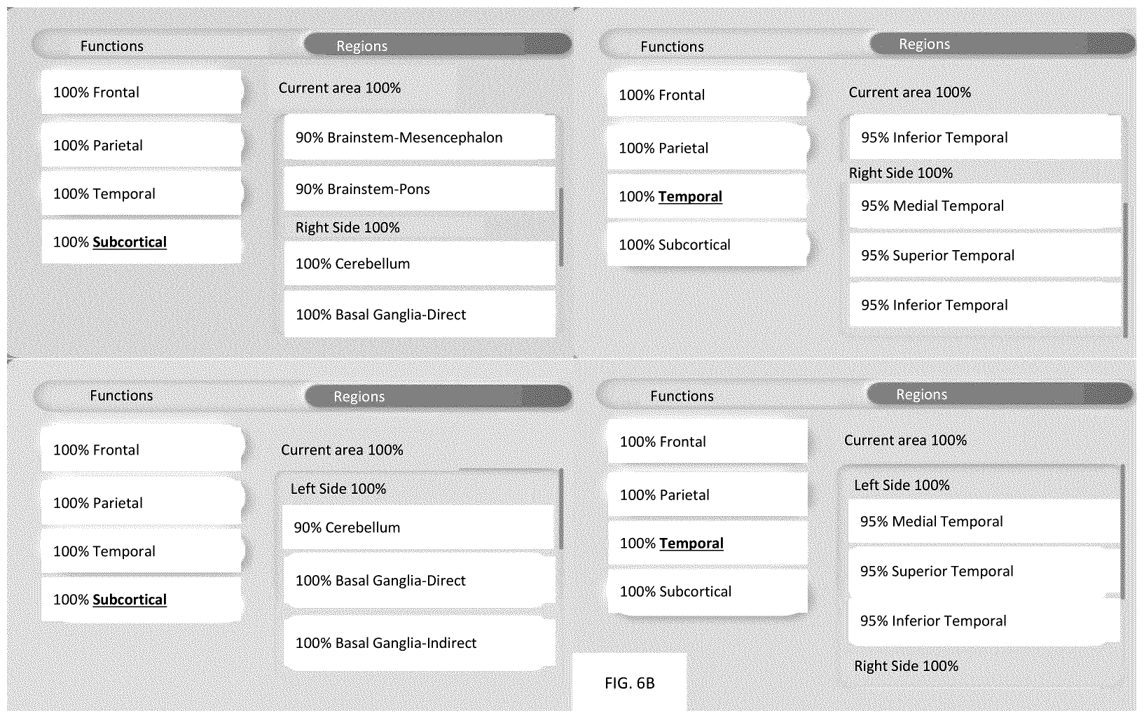

Reference is now made to FIGS. 6A and 6B which present data that may be accumulated from a subject, representing a use case of an embodiment of the invention. The information depicted in FIGS. 6A and 6B may be presented via output device 8 (e.g., UI 50), and may serve a care giver or physician to obtain an overview of a patient's condition.

In the example of FIGS. 6A and 6B, it is clear that the main regions of interest are the parietal lobes on both sides, the left cerebellum, the frontal lobe on the right (mainly the dorsolateral prefrontal cortex (DLPFC)) as well as the indirect pathway of the basal ganglia on the right. It may also be beneficial to activate the midbrain and the pons of the brainstem, and the right temporal lobes, although the latter appears to be of secondary importance.

Based on these selected regions 70SR, system 100 may prompt the patient to play the following games 80G, having the associated parameters 80P:

A first game 80G may aim to find and identify whether a presented object (e.g., a body part) belongs to the right or left side. Parameters 80P of the game may dictate that the background for the game will be texturized, with cold (e.g., bluish) hues. At high levels of the game, objects will be presented as rotating in a clockwise direction.

Rationale for selecting these parameters 80P includes: (a) The skill of determining right from left is linked to the left parietal lobe; (b) cold background hues affect the right brain hemisphere more than the left one; (c) The task of tracing an object over texturized background activates the parietal lobe more than the temporal lobe; and (d) Clockwise rotation simulates a movement of the head in the opposite direction, thus activating the vestibular system and the cerebellum on the left hemisphere.

A second game 80G may have a goal of matching facial expressions (e.g., an image of a smiling child) to a word expressing emotion (e.g., “happy”). Parameters 80P of the game may dictate that the background for the game will be texturized and have cold hues. At high levels of the game, objects will be presented as rotating in a clockwise direction. Movement of objects across the screen will be smooth (without jitters), and will include horizontal (left to right), diagonal (top left to bottom right), and vertical (top to bottom) motions. The emotion words will be presented at the bottom left of the UI 50 screen.

Rationale for selecting these parameters 80P includes: (a) Recognition of facial expressions, as part of non-verbal communication, is associated with the right hemisphere (b) Horizontal movement from left to right is associated with right parietal/temporal lobes. It is also closely related to the pons of the brain stem. (c) Vertical movement from top to bottom is related to the right hemisphere, as well as the middle brain (mesencephalon) of the brain stem. (d) Diagonal movement from top-left to bottom-right is related to the right hemisphere, as well as to the left cerebellum. (e) Placement of objects on the screen will be mainly on the left side of the screen.

A third game 80G may present a goal of finding objects that had previously appeared in a sequence of images shown on the screen. This game will examine a patient's working memory, control of saccadic eye movements, and ability to focus vision. Parameters 80P of the game may dictate that direction of movement of the objects on the screen will be right to left and/or bottom-right to top-left.

Rationale for selecting these parameters 80P is as follows: (a) Saccadic eye movements are related to the frontal lobes of the same side as that which the movement originates. (b) Stopping the saccadic jitters is mainly related to the cerebellum, on the same side as that of the direction of movement. Pertaining to this example, the stimulated lobes would be the right frontal lobe and the left cerebellum.

A fourth game 80G may require visual inversion capabilities. In this game, a user will need to determine what objects would look like subsequent to their rotation. This game will activate the user's skill of spatial awareness. Parameters 80P of the game may dictate a background as described above, and show complex objects such as animals.

Rationale for selecting these parameters 80P is as follows: (a) Visual inversion is mainly associated with the parietal lobes, especially on the right hemisphere. (b) Spatial awareness is related to normal functioning of the cerebellum.

A fifth game 80G may require the user to identify characters on the screen, match them to characters on a side of the screen, and either click, or not click on the characters according to the match result. This game will evoke two main components: optokinetic capability, and a Go/No-go activity. Optokinetic activity is related to the parietal lobes, where horizontal representation is related to the pons, and vertical representation is related to the midbrain. Go/No-Go activity requires efficient activity of the indirect pathway of the basal ganglia. Parameters of backgrounds and object representation will be as described above. At high levels of the game, a component of visual inversion as described above may be added.

A test case illustrating the operation of system 100 may include a clinical assessment and treatment process as follows:

Evaluation module 40 may receive input data for a subject, which may include an expert diagnosis 20ED, such as Autism Spectrum Disorder (ASD), which may be associated with right hemisphere deficit, and a profile data elements 20PR, such as age and gender. Evaluation module 40 may further obtain (e.g., via UI 50) results from an initial questionnaire 410QU, which may indicate reduced functionality in various brain regions, including the right dorsolateral prefrontal cortex (DLPFC), right orbital frontal cortex, right parietal and inferior parietal regions, right basal ganglia indirect pathway, and left cerebellum.

Based on the input data, activity selection module 400 may select, or update a list of assessment games 410GA. Brain assessment module 30 may determine a suspicion of right hemisphere deficit with cerebellar involvement, and reflect that deficit in the functional region map 20FRM of a personalized (e.g., pertaining to the specific subject) brain map 20.

Evaluation module 40 may then initiate an assessment phase by prompting the subject to engage in assessment games 410GA via UI 50. The assessment games may include:

A first game 410GA-1 of hitting targets on the screen. This game may focus on distinguishing right from left hemisphere function and cerebellar involvement, as represented in the region stimulus map 20RSM of brain map 20. Game monitoring module 90 may consider correlations between brain regions and corresponding stimuli in region stimulus map 20RSM, to monitor and analyze game performance parameters 90GP such as: (a) success rate with various changes to game parameters 80P; (b) Target placement on the screen, where targets on the left may challenge the right hemisphere more, as indicated by region stimulus map 20RSM; (c) timing and accuracy of contact to assess cerebellar function (d) reaction time to assess frontal lobe function; and (e) movement direction to assess parietal/temporal lobe involvement.

A second game 410GA-2 of pursuing a target on the screen. This game may focus on challenging parietal vs temporal lobe function. Game monitoring module 90 may consider correlations between brain regions and corresponding stimuli in region stimulus map 20RSM, to monitor, and analyze game performance parameters 90GP such as: (a) Smoothness of pursuit over textured vs solid backgrounds; and (b) Vertical vs horizontal movement challenges (thereby assessing pons vs mesencephalon functionality).

Activity selection module 400 may continue the assessment phase with increasing game complexity and parameter changes 80P. Additional game parameters 80P and performance parameters 90GP that may be evaluated include (a) Go/No-go tasks; (b) Smooth vs non-smooth movement, requiring smooth pursuit or saccadic eye movements; (c) Obstacle and target placement in various visual field quadrants; (d) Positioning of distractors, having various auditory and/or visual properties, and the like.

Additionally or alternatively, game monitoring module 90 may collect and analyze various game performance parameters 90GP during the assessment phase, including for example (a) Success rates with various parameters; (b) Timing of responses (e.g., early or late clicks); (c) Accuracy of responses (e.g., clicking at the correct location); (d) Indicators of hyperactivity/impulsivity (e.g., excessive mouse clicks); (e) Indicators of hypoactivity (e.g., missed mouse clicks); and (f) Reaction times.

Upon completion of the assessment phase, brain assessment module 30 may generate a profile of the subject's brain function, identifying areas of reduced functionality as represented in the functional region map 20FRM of brain map 20. Based on this profile of brain map 20, game generation module 80 may create personalized curative games 80G designed to challenge, or stimulate the identified areas of reduced functionality, utilizing the associations between brain regions and audiovisual stimuli specified in the region stimulus map 20RSM. Game generation module 80 may calibrate the difficulty level of these curative games 80G to match the subject's performance level in the assessment games 410GA. Additionally, or alternatively, the difficulty level of these curative games 80G may dynamically evolve over time, as a function of a rate of change 30RC (e.g., improvement) in one or more parameters of game performance 90GP. For example, a speed of the pursued target may increase between games, when a game performance parameter 90GP such as a number of unnecessary mouse clicks is improved (e.g., in this case-reduced).

System 100 may thereby provide an appropriate, dynamically changing level of challenge for the subject's current capabilities, as reflected in the subject's personalized brain map 20.

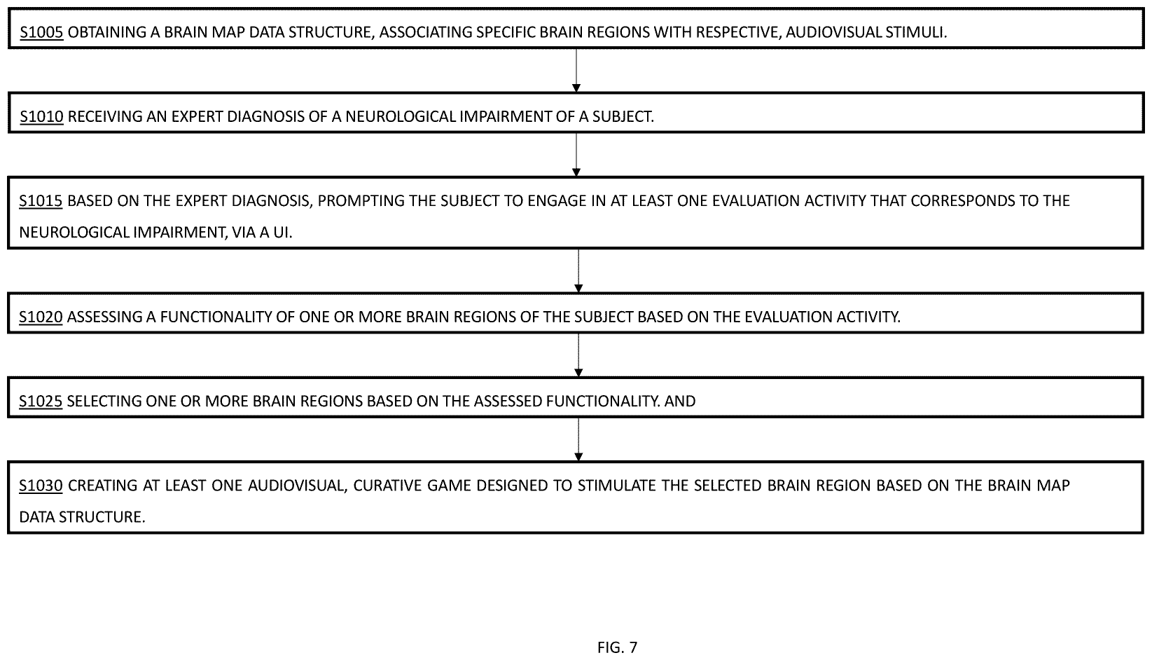

Reference is now made to FIG. 7, which is a flow diagram, depicting a method of creating curative games by at least one processor, according to some embodiments of the invention.

As shown in step S1005, the at least one processor (e.g., processor 2 of FIG. 1) may obtain a brain map data structure (e.g., 20 of FIGS. 2, 4A, 4B), associating specific brain regions with respective, audiovisual stimuli.

As shown in steps S1010 and S1015, the at least one processor 2 may receive an expert diagnosis (e.g., 20ED of FIG. 2) of a neurological impairment of a subject. Based on the expert diagnosis, the at least one processor may prompt the subject to engage in at least one evaluation activity (e.g., 410EV of FIG. 2) that corresponds to the neurological impairment, e.g., via a UI (e.g., UI 50 of FIG. 2).

As shown in steps S1020 and S1025, the at least one processor 2 may calculate or assess a functionality of one or more brain regions of the subject based on the evaluation activity 410EV, and select (e.g., 70SR of FIG. 2) one or more brain regions based on the assessed functionality.

As shown in step S1030, the at least one processor 2 may subsequently create at least one audiovisual, curative game (e.g., 80G of FIG. 2) designed to stimulate the selected brain region 70SR, based on the brain map data structure 20.

Embodiments of the invention may include a practical application in the technological field of assistive diagnostics and treatment of neurological impairments. As elaborated herein, embodiments of the invention may automatically identify regions in a user's (e.g., a “subject” or “patient”) brain that perform poorly (e.g., is attributed a low score 35D/30AS), and devise personalized, curative stimuli (e.g., games 80G) for activating those specific regions. Embodiments of the invention may iteratively adjust the stimuli 80G, in a repetitive and dynamic manner, to accustom the evolution or change in the user's performance of the presented games 80G.

Unless explicitly stated, the method embodiments described herein are not constrained to a particular order or sequence. Furthermore, all formulas described herein are intended as examples only and other or different formulas may be used. Additionally, some of the described method embodiments or elements thereof may occur or be performed at the same point in time.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

Various embodiments have been presented. Each of these embodiments may of course include features from other embodiments presented, and embodiments not specifically described may include various features described herein.

1. A method of producing a curative game by at least one processor, the method comprising:
obtaining a brain map data structure, associating specific brain regions with respective, audiovisual stimuli;
receiving an expert diagnosis of a neurological impairment of a subject;
based on the expert diagnosis, prompting the subject to engage in at least one evaluation activity that corresponds to the neurological impairment, via a user interface (UI);
assessing a functionality of one or more brain regions of the subject based on the evaluation activity and brain map data structure;
selecting one or more brain regions based on the assessed functionality; and
creating at least one audiovisual, curative game designed to stimulate the selected brain region based on the brain map data structure.

2. The method of claim 1, wherein assessing said functionality is performed iteratively, wherein each iteration comprises:
prompting the subject to engage in an evaluation activity;
assessing a functionality of one or more brain regions of the subject based on outcome of the evaluation activity;
updating the brain map data structure, based on the assessed functionality, thereby producing a personalized brain map data structure; and
employing a decision tree, to select an evaluation activity for a subsequent iteration, based on the personalized brain map data structure.

3. The method of claim 2, wherein the evaluation activity comprises a questionnaire, and wherein the method further comprises:
presenting the questionnaire to the subject via the UI;
obtaining the subject's response to the questionnaire via the UI;
assessing the functionality of the one or more brain regions of the subject based on the subject's response and brain map; and
personalizing the brain map to reflect the assessed functionality of one or more brain regions of the subject.

4. The method of claim 2, wherein the evaluation activity comprises an evaluation game, and wherein the method further comprises:
presenting the evaluation game to the subject via the UI;
monitoring the subject's playing, to extract a value of at least one evaluation game performance parameter;
assessing the functionality of the one or more brain regions of the subject based on the at least one evaluation game performance parameter; and
personalizing the brain map to reflect the assessed functionality of one or more brain regions of the subject.

5. The method of claim 1, wherein creating a curative game comprises:
selecting, from a pool of game templates, a game template that corresponds to the neurological impairment; and
adapting at least one game parameter of the selected game template, so as to stimulate the selected brain region, based on the personalized brain map data structure.

6. The method of claim 5, further comprising an iterative process, wherein each iteration comprises:
prompting the subject to play, via the UI, one or more iterations of at least one curative game;
monitoring the subject's playing, to extract a value of at least one game performance parameter in the one or more iterations;
analyzing the game performance parameter value, to identify suboptimal functionality of the one or more selected brain regions; and
updating the brain map data structure to reflect the identified suboptimal functionality of the one or more selected brain regions.

7. The method of claim 6, wherein identifying suboptimal functionality comprises:
determining a rate of change across the one or more iterations, of function of a specific region of the one or more selected brain regions; and
analyzing the determined rate of change in view of a predetermined baseline rate of expected change, to identify the suboptimal functionality.

8. The method of claim 6, further comprising adjusting at least one game parameter of the created game based on the brain map data structure, so as to stimulate the selected brain region of the identified suboptimal functionality.

9. The method of claim 5, further comprising:
selecting, from a pool of game templates, a specific game template that corresponds to the neurological impairment; and
adapting at least one game parameter of the selected game template based on the brain map data structure, so as to stimulate the selected brain region of the identified suboptimal functionality.

10. The method of claim 6, further comprising:
obtaining a pretrained machine learning (ML) based model; and
inferring the ML based model on (i) the one or more game performance parameters, and (ii) the expert diagnosis, to predict a refined diagnosis of the neurological impairment of the subject.

11. The method of claim 6, further comprising:
determining a rate of change across the one or more iterations, of functionality of a specific region of the one or more selected brain regions; and
inferring an ML-based model on (i) the determined rate of change, and (ii) the expert diagnosis, to predict a refined diagnosis of the neurological impairment of the subject.

12. The method of claim 6, further comprising:
obtaining a pretrained ML-based model;
inferring the ML based model on the one or more game performance parameters, to predict suboptimal functionality of at least one brain region of the subject; and
updating the brain map data structure to reflect the predicted suboptimal functionality of the at least one brain region.

13. The method of claim 5, wherein creating a curative game further comprises:
obtaining at least one profile data element, representing a profile of the subject;
obtaining at least one medical data element, representing a medical history of the subject; and
adapting a game parameter of the selected game template based on the at least one profile data element and the at least one medical data element, so as to stimulate the selected brain region, according to the brain map data structure.

14. A system for treating a neurological impairment of a subject, the system comprising a non-transitory memory device, wherein modules of instruction code are stored, and at least one processor associated with the memory device, and configured to execute the modules of instruction code, whereupon execution of said modules of instruction code, the at least one processor is configured to:
obtaining a brain map data structure, associating specific brain regions with respective, audiovisual stimuli;
receiving an expert diagnosis of a neurological impairment of a subject;
based on the expert diagnosis, prompting the subject to engage in at least one evaluation activity that corresponds to the neurological impairment, via a user interface (UI);
based on the evaluation, producing an assessment of functionality of one or more brain regions of the subject;
selecting one or more brain regions based on said assessment;
creating at least one audiovisual, curative game designed to stimulate the selected brain region based on the brain map data structure; and
prompting the subject to play the created game via the UI.

15. The system of claim 14, wherein the at least one processor is configured to prompt the subject to engage in an evaluation activity by:
presenting a questionnaire to the subject via the UI;
obtaining the subject's response to the questionnaire via the UI;
assessing the functionality of the one or more brain regions of the subject based on the subject's response; and
personalizing the brain map to reflect the assessed functionality of one or more brain regions of the subject.

16. The system of claim 14, wherein the at least one processor is configured to prompt the subject to engage in an evaluation activity by:
presenting evaluation game to the subject via the UI;
monitoring the subject's playing, to extract a value of at least one evaluation game performance parameter;
assessing the functionality of the one or more brain regions of the subject based on the at least one evaluation game performance parameter; and
personalizing the brain map to reflect the assessed functionality of one or more brain regions of the subject.

17. The system of claim 14, wherein the at least one processor is configured to create the at least one curative game by:
selecting, from a pool of game templates, a game template that corresponds to the neurological impairment; and
adapting at least one game parameter of the selected game template, so as to stimulate the selected brain region, based on the brain map data structure.

18. The system of claim 14, wherein the at least one processor is configured to implement an iterative process, wherein each iteration comprises:
prompting the subject to play, via the UI, one or more iterations of at least one created game;
monitoring the subject's playing, to extract a value of at least one game performance parameter in the one or more iterations;
analyzing the game performance parameter values, to identify suboptimal functionality of the one or more selected brain regions; and
updating the brain map data structure to reflect the identified suboptimal functionality of the one or more selected brain regions.

19. The system of claim 18, wherein the at least one processor is configured to identify suboptimal functionality by:
determining a rate of change across the one or more iterations, of function of a specific region of the one or more selected brain regions; and
analyzing the determined rate of change in view of a predetermined baseline rate of expected change, to identify the suboptimal functionality.

20. The system of claim 18, wherein the at least one processor is configured to adjust at least one game parameter of the created game based on the brain map data structure, so as to stimulate the selected brain region of the identified suboptimal functionality.
